(19) United States
(12) Patent Application Publication
(10) Pub. No.: US 2005/0124018 A1
(43) Pub. Date: Jun. 9, 2005

(54) Method of measuring ATP by radiating ultraviolet light and apparatus using the same

(75) Inventor: Hyo-bong Hong, Gyeonggi-do (KR)

Correspondence Address: BURNS DOANE SWECKER & MATHIS L L P, POST OFFICE BOX 1404, ALEXANDRIA, VA, 22313-1404, US

(73) Assignee: Samsung Electronics Co., Ltd., Gyeonggi-do (KR)

(21) Appl. No.: 10/960,685

(22) Filed: Oct. 8, 2004

(30) Foreign Application Priority Data
Oct. 9, 2003 (KR) 10-2003-0070272

(51) Int. Cl. C12Q 1/66

(52) U.S. Cl. 435/8

(57) ABSTRACT

Provided are a method of measuring an adenosine 5′-triphosphate (ATP) and an apparatus for analyzing an ATP using the same. The method comprises treating a sample such that an ATP that may be present in the sample can chemically react; reacting an ATP-reactive mixture comprising a luciferin, a luciferase and a cation with the treated sample under oxygen; and generating photoluminescence at a wavelength range of 475 to 675 nm by amplifying luminescence generated by the reaction with ultraviolet light in a wavelength range of 320 to 370 nm.

### BACKGROUND OF THE INVENTION

This application claims the priority of Korean Patent Application No. 2003-70272, filed on Oct. 9, 2003, in the Korean Intellectual Property Office, the disclosure of which is incorporated herein in its entirety by reference.

1. Field of the Invention

The present invention relates to a method of measuring an adenosine 5′-triphosphate (ATP) and an apparatus for analyzing an ATP using the same, and more particularly, to a method of measuring an ATP to determine whether a contaminating material was derived from a living organism and an apparatus for analyzing an ATP using the same.

2. Description of the Related Art

Determining whether a source of a contaminating material existing in an electronic product, a precise machine, fine chemistry, a hospital, etc. in a very small amount is a microorganism or just an organic material is very important because the method of removing the source depends on the type of contamination. Methods of determining whether a contaminating material is a living organism have been divided into three main methods. The first method is to measure growth of a microorganism or to observe an actual shape thereof by using a microscope (optical or electron beam microscope). The second method is to analyze a specific metabolite of a microorganism, thereby determining whether the microorganism has grown. The third method is to analyze biological material such as a protein or a DNA.

The third method is used to confirm the presence of a living organism by measuring presence of an adenosine 5′-triphosphate (ATP), in which is a known energy carrier of all living organisms. This is considered to be a very sensitive and reliable method, and has been used since 1947. Accordingly, the method has been applied in many fields.

U.S. Pat. No. 3,933,592 discloses a method of detecting ATP. The method is carried out by measuring luminescence generated by a reaction represented by reaction scheme I below involving a luciferin and a luciferase:

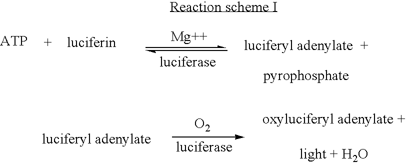

Reactants required in the luminescent reaction are a luciferin as a substrate, a luciferase as an enzyme, an ATP as an activating agent, a cation (mainly, magnesium) and an oxygen. The overall reaction is an oxidation reaction in which enzymic luciferase functions as a catalyst, and luminescence is generated. Such a reaction is specific to an ATP, which cannot be substituted by any other chemical material. Furthermore, an ATP is present in all living organisms, and thus the presence of a microorganism can be quickly determined from the reaction.

As a method of measuring luminescence of the ATP-specific luminescent reaction, a method using a luminometer has been most commonly used and is known to be a reliable method.

However, the most serious drawback of the method of measuring an ATP is that a sample should always be in a liquid state, a probability that errors may occur in sampling process is high, and an analysis process takes a relatively long time. Further, when an ATP is present in a very small amount, the strength of luminescence is so weak that measurement sensitivity may decrease.

Particularly, when the method of measuring an ATP is applied to a solid sample, it is difficult to perform sampling, it is almost impossible to apply the general biochemical reaction described above due to an extremely low concentration of microorganisms, and analyzing takes a long time.

In order to measure an ATP on a solid surface such as an electronic product by using the method of measuring an ATP, a method illustrated in FIG. 1 is generally performed. However, if an ATP is measured by such a method, when a contaminating region is very small but an overall sample is very large as in an LCD substrate or a semiconductor, information concerning luminescence obtained from a luminescence analyzer, if any, is limited to a spectrum, and thus it is difficult to determine what region of an overall sample is contaminated. That is, since the contaminated region itself cannot be confirmed, analysis concerning what region is contaminated is almost impossible. Accordingly, in order to identify specifically what region is biologically contaminated, a new technique is required.

### SUMMARY OF THE INVENTION

The present invention provides a method of measuring a presence of, an amount of and a position of an ATP on a solid surface such as an LCD substrate or a semiconductor surface by employing a luminescent reaction using a luciferin.

The present invention also provides an apparatus for analyzing an ATP that can measure a presence of, an amount of and a position of an ATP on a solid surface by employing the method.

According to an aspect of the present invention, there is provided a method of measuring an adenosine 5′-triphosphate (ATP) comprising: treating a sample such that an ATP that may be present in the sample can chemically react; reacting an ATP-reactive mixture comprising a luciferin, a luciferase and a cation with the treated sample under oxygen; and generating photoluminescence at a wavelength of 475 to 675 nm by amplifying luminescence generated by the reaction with ultraviolet light in a wavelength range of 320 to 370 nm.

The generated photoluminescence may be photographed with a photo luminescent photographic apparatus. Further, the photograph may be treated using an image treating apparatus, thereby identifying a position of an ATP in a sample.

The sample may be in a liquid or a solid state in the method of measuring an ATP.

In the method, the operation of treating the sample can be achieved by, extracting an ATP present in a microorganism. Such extraction of an ATP may be achieved by treating with a benzalkonium chloride or heat-treating.

Further, the operation of treating the sample may include reacting the sample with a phosphorylating enzyme to convert an ATP precursor present in the sample into an ATP. A phosphocreatine kinase can be used as the phosphorylating enzyme.

Magnesium, potassium or sodium, etc. may be used as the cation. Most preferably, magnesium can be used.

According to another aspect of the present invention, there is provided an apparatus for analyzing an ATP that can measure a presence of, an amount of and a position of an ATP, and combinations thereof by employing the method.

### BRIEF DESCRIPTION OF THE DRAWINGS

The above and other features and advantages of the present invention will become more apparent by describing in detail exemplary embodiments thereof with reference to the attached drawings in which:

FIG. 1 is a flowchart illustrating conventional a method of measuring an ATP on a solid surface;

FIG. 2 is a flowchart illustrating a method of measuring an ATP according to an embodiment of the present invention;

FIG. 3 is a schematic diagram illustrating an apparatus for analyzing an ATP and the principle thereof according to an embodiment of the present invention;

FIG. 4 is a spectrum of a sample generated when ultraviolet light in a wavelength range of 320 to 370 nm is irradiated on the sample when measuring an ATP according to an embodiment of the present invention;

FIG. 5 illustrates spectrums of a sample and a control generated when ultraviolet light in a wavelength range of 320 to 370 nm is irradiated on the sample and control when measuring an ATP according to the present invention;

FIG. 6 is a photograph of a filter taken by a fluorescent camera when a sample was filtered according to a method of measuring an ATP according to an embodiment of the present invention;

FIG. 7A is a photograph taken by a fluorescent camera when a microorganism was inoculated into an LCD color filter according to a method of measuring an ATP according to an embodiment of the present invention;

FIG. 7B is a photograph taken by a fluorescent camera of an LCD color filter of which analysis was asked by a relevant company according to a method of measuring an ATP according to an embodiment of the present invention; and

FIG. 8 is a photograph of a filter taken by a fluorescent camera when a sample was filtered according to a method of measuring an ATP according to an embodiment of the present invention except using ultraviolet light in a wavelength range of 200 to 320 nm instead of 320 to 370 nm.

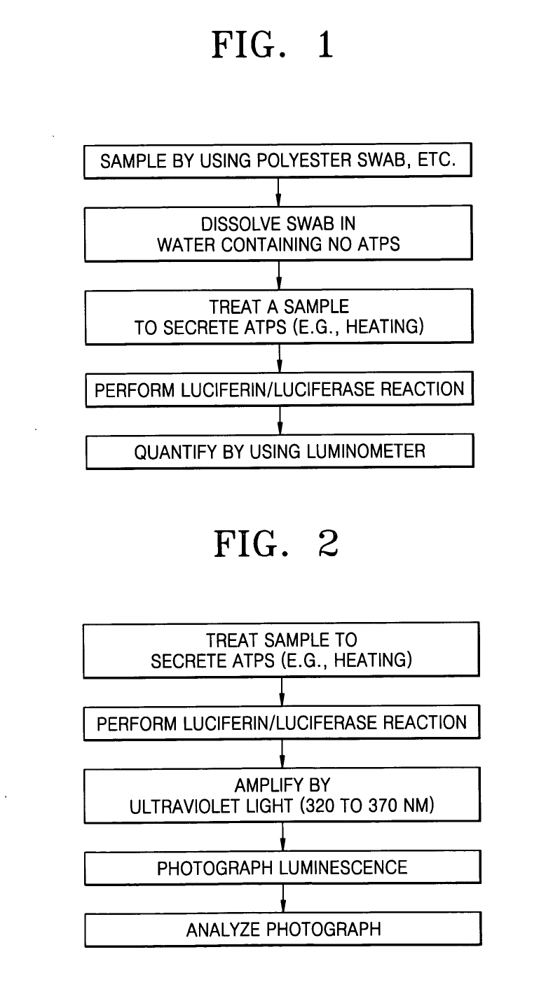

### DETAILED DESCRIPTION OF THE INVENTION

A method of measuring an ATP of the present invention comprises treating a sample such that an ATP that may be present in the sample can chemically react; reacting an ATP-reactive mixture comprising a luciferin, a luciferase and a cation with the treated sample under oxygen; and generating photoluminescence at a wavelength range of 475 to 675 nm by amplifying luminescence generated by the reaction with ultraviolet light in a wavelength range of 320 to 370 nm.

According to a conventional method of measuring an ATP, luminescence generated in the reaction is directly measured by a luminescence analyzer. However, in the present invention luminescence generated in the reaction is amplified with ultraviolet light having a predetermined wavelength, thereby enhancing the sensitivity of measuring an ATP and enabling photographing of an image with a photoluminescent photographic apparatus. Accordingly, the position of a region contaminated with microorganisms having ATPs can be determined.

The method of measuring an ATP can be applied to a solid sample or a liquid sample. The method of measuring an ATP is particularly useful over a conventional method when being used on a solid sample. As described above, for a solid sample, the luminescence generation and luminescence amplification by ultraviolet light are directly performed on the solid sample, and then the solid sample is photographed by a photoluminescent photographic apparatus to identify a luminescent position. For a liquid sample, the sensitivity of measuring an ATP is improved since the generated luminescence is amplified by ultraviolet light, thereby enhancing the strength of luminescence.

In order to generate a luminescent reaction in a sample using luciferin in the method of measuring an ATP according to the present invention, an ATP should be extracted so that an ATP that may be present in a sample can react. Examples of a method of extracting an ATP include a method of treating a microorganism with a benzalkonium chloride, a method using heat treatment, etc. The sample can be directly heated with a heating coil or a similar heating device in the heat treatment. For a solid sample, hot water can be applied to the surface of the solid sample, and for a liquid sample, hot water can be directly added to the liquid sample. The temperature of a sample can be raised to about 95 to 100□ by using the heat treatment.

In the method of measuring an ATP, magnesium, potassium or sodium, etc. can be used as the cation used in the reaction for generating luminescence. Most preferably, magnesium can be used.

The method of measuring an ATP is illustrated in FIG. 2.

The present invention also provides an apparatus for analyzing an ATP that can measure a presence of, an amount of and a position of an ATP by employing the method.

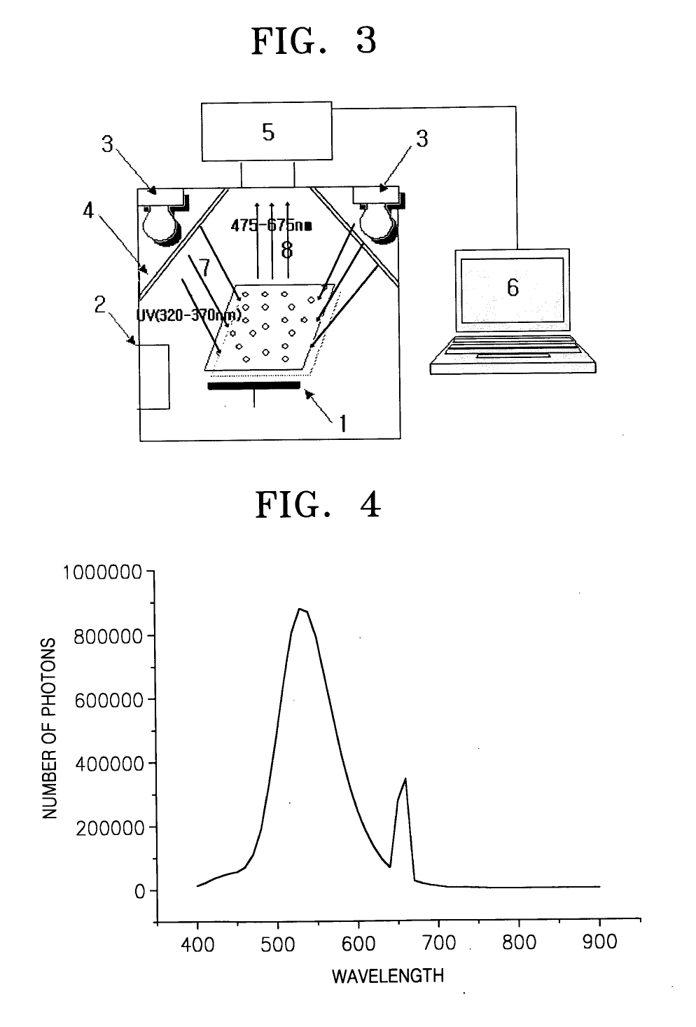

Referring to FIG. 3, the apparatus for analyzing an ATP comprises a sample-treating region 1 in which a sample is treated such that an ATP of a sample can chemically react; a reagent-feeding region 2 through which a reagent that reacts with an ATP in a sample to generate luminescence is provided to the sample on the sample-treating region; a lighting region 3 generating ultraviolet light in a wavelength range of 320 to 370 nm to amplify luminescence generated in the reaction of the reagent with the sample; a filter 4 transmitting ultraviolet light only in a wavelength range of 320 to 370 nm; a photoluminescent photographic apparatus 5 photographing a photoluminescence generated by the amplification of the luminescence; and an image analyzing apparatus 6 analyzing the photographed luminescent image.

The lighting region 3 is a light source that can generate light with a wavelength of 320 to 370 nm, such as a halogen lamp, a tungsten lamp or a mercury lamp, etc.

The photoluminescent photographic apparatus can be a CCD camera or a fluorescent camera.

The deposition of the apparatus for analyzing an ATP and the principle thereof according to an embodiment of the present invention are schematically illustrated in FIG. 3.

The present invention will now be described in greater detail with reference to the following examples. The following examples are for illustrative purposes only and are not intended to limit the scope of the invention.

### EXAMPLES

### Experimental Example 1

### Determining Absorbing Wavelength

A specific wavelength, at which a luminescence generated in a luciferin/luciferase luminescent reaction can be absorbed and then amplified, was measured by using an ultraviolet light-visible light spectrophotometer. First, an ATP and luciferin/luciferase were mixed with a ratio of 1:10, and then 100 □ of the mixed solution was diluted with ultrapure water to a final volume of 1 □. Absorbance of the diluted solution was measured using light in a wavelength range of 200-900 nm. A luciferin/luciferase solution not containing an ATP was used as a control.

The result of measurement indicated that light absorbed by the luminescence produced in a luciferin/luciferase luminescent reaction was ultraviolet light in a wavelength range of 320 to 370 nm.

### Experimental Example 2

### Analysis of Luminescence with a Wavelength Range of 320 to 370 nm

A region where photoluminescence was generated was confirmed by light in a wavelength range of 320 to 370 nm. A specific wavelength in the range of 320 to 370 nm was fixed, as an excitation wavelength, on luminescence generated in a diluted solution of an ATP and luciferin/luciferase (1:10) in the same manner as in the experimental example 1. Then, the intensity of light in a wavelength excited at a wavelength range of 200 to 900 nm was measured. A luciferin/luciferase solution not containing an ATP was used as a control, as in the experimental example 1.

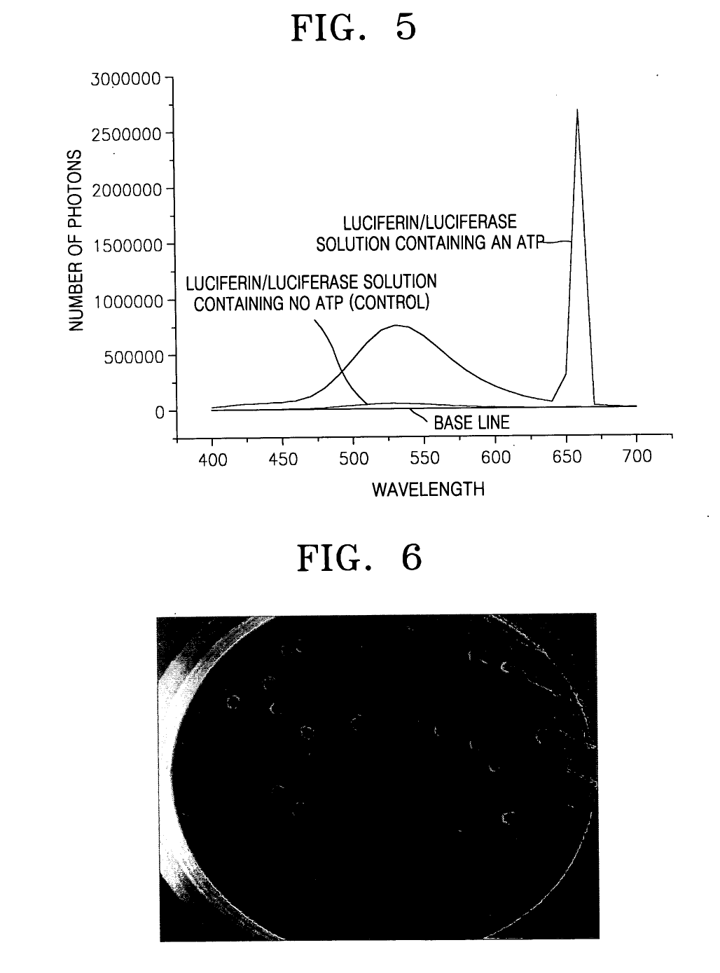

It was found that the photoluminescence which resulted from the excitation of the luminescence was in the visible range with a wavelength of 475 to 675 nm. The results are shown in graphs of FIGS. 4 and 5.

### Example 1

### Construction of Apparatus for Analysing ATP

An apparatus for analysing an ATP comprised a sample treating region, a reagent feeding region containing luciferin/luciferase, an Arc light source, a filter transmitting only ultraviolet light in a wavelength range of 320 to 370 nm of the light source, a fluorescent camera (OPTRONICS DEI-470) that can photograph generated photoluminescence, and a computer image analyzing apparatus (Zeiss KS-400 ver 3.0) that can analyze an image photographed by a fluorescent camera.

### Example 2

### ATP Analysis Using Standard Microorganism

Two types of bacterial strains were isolated from water during preparation process and identification analysis thereof were carried out by Korea Research Institute of Bioscience and Biotechnology (KRIBB) prior to such bacterial strains being used. From the result of 16sRNA analysis, it was determined that one strain had 99.8% of a family relation to a standard bacterial strain (IAM 12605T) of Bacillus cereus, and the other strain had 98.3% of a family relation to a standard bacterial strain (DSM654T) of Psudomonas saccharophila. These strains were grown in nutrient broths, diluted to a 10⁻⁷ ratio, and then filtered, or a single colony was taken on solid state, and placed on an LCD color filter.

For the filtered sample, a benzalkonium chloride (10 mM) was sprayed on the entire surface of the filter using a spray for TLC, and then a luciferin/luciferase solution was sprayed on the entire surface of the filter.

For the LCD color filter sample, 50 □ of ultrapure water heated to 100□ was applied to a region on the filter, and then the sample was left for about 1 minute, and then 50 □ of 10 mM benzalkonium chloride was placed on the filter and the sample was left for about 1 minute.

100 □ of D-luciferin/luciferase solution was added to the latter sample, and then resulting luminescence was excited with an ultraviolet light at a wavelength of 365 nm using an Arc light source and a filter, and a photoluminescent image was photographed with a fluorescent camera, and analysed using an image analyzing apparatus (OPTRONICS DEI-470, Zeiss KS400 ver3.0).

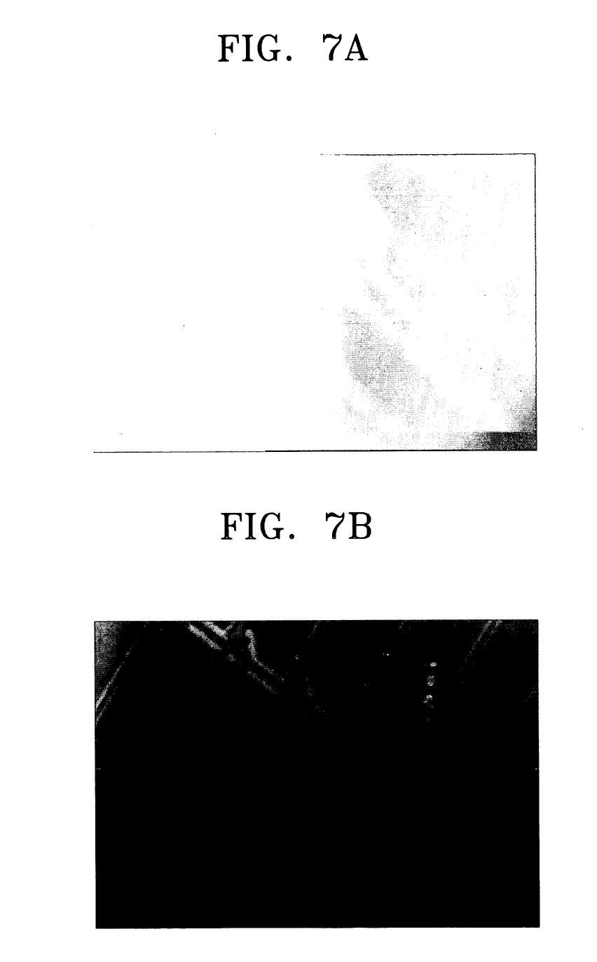

The photographed image of the filtered sample is shown in FIG. 6, and the photographed image of the sample on the LCD color filter is shown in FIG. 7A. In FIGS. 6 and 7A, it can be seen that a region on which a microorganism existed produced fluorescence.

### Example 3

### ATP Analysis Using Actual Sample

An LCD color filter of which analysis was asked by a relevant company was treated according to the same manner as in example 2, and then analysed.

The results are shown in FIG. 7B. In FIG. 7B, it can be seen that the LCD color filter of which analysis asked by the relevant company was not contaminated with a microbial foreign material since a luminescent reaction did not occur.

### Comparative Example 1

### ATP Analysis in Other Wavelength Region

ATP analysis was performed in the same manner as in example 1 except that a filter that transmits ultraviolet light in a wavelength range of 200 to 320 nm was used. The filtered sample of example 2 was used as a sample.

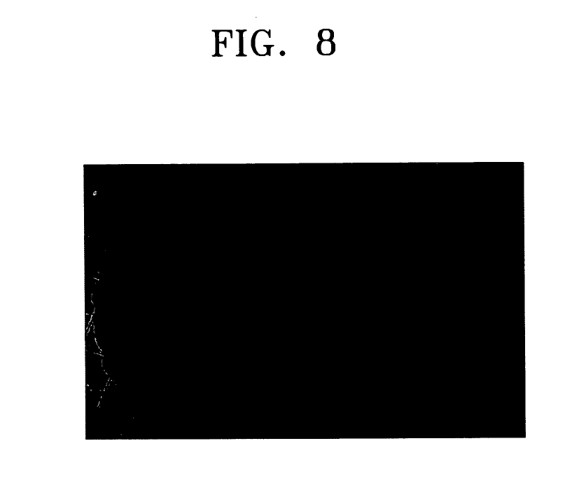

The results are shown in FIG. 8. As shown in FIG. 8, there was no fluorescence.

As described above, according to the present invention, it can be confirmed whether a foreign material on a solid surface such as a LCD or a semiconductor is a biological material. When the surface area to be analyzed is so very large, even when contamination of the overall sample cannot be determined easily with a light microscope or an electronic microscope, the method of the present invention can be reliably used to determine the type of contamination and its location. Further, very fast in-situ analysis can be possible since a special sampling process is not needed.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of measuring an adenosine 5′-triphosphate (ATP) comprising:
treating a sample such that an ATP that may be present in the sample can chemically react;
reacting an ATP-reactive mixture comprising a luciferin, a luciferase and a cation with the treated sample under oxygen; and
generating photoluminescence at a wavelength range of 475 to 675 nm by amplifying luminescence generated by the reaction with ultraviolet light in a wavelength range of 320 to 370 nm.

2. The method of measuring an ATP of claim 1, wherein the generated photoluminescence is photographed by a photoluminescent photographic apparatus.

3. The method of measuring an ATP of claim 2, wherein the photograph is analyzed by using an image analyzing apparatus, thereby identifying a position of an ATP in a sample.

4. The method of measuring an ATP of claim 1, wherein the sample is a solid.

5. An apparatus for analyzing an ATP capable of measuring one selected from the group consisting of a presence of, an amount of, and a position of an ATP in a sample, and combinations thereof, according to the method of claim 1, and comprising:
a sample-treating region in which a sample is treated such that an ATP of a sample can chemically react;
a reagent-feeding region through which a reagent that reacts with an ATP in a sample to generate luminescence is provided to the sample on the sample-treating region;
a lighting region generating ultraviolet light in a wavelength range of 320 to 370 nm to amplify luminescence generated in the reaction of the reagent with the sample;
a filter transmitting ultraviolet light only in a wavelength range of 320 to 370 nm;
a luminescent photographic apparatus photographing a photoluminescence generated by the amplification of the luminescence; and
an image analyzing apparatus analyzing the photographed photoluminescent image.

6. The apparatus for analyzing an ATP of claim 5, wherein the photoluminescent photographic apparatus is a CCD camera or a fluorescent camera.

7. An apparatus for analyzing an ATP capable of measuring one selected from the group consisting of a presence of, an amount of, and a position of an ATP in a sample, and combinations thereof, according to the method of claim 2, and comprising:
a sample-treating region in which a sample is treated such that an ATP of a sample can chemically react;
a reagent-feeding region through which a reagent that reacts with an ATP in a sample to generate luminescence is provided to the sample on the sample-treating region;
a lighting region generating ultraviolet light in a wavelength range of 320 to 370 nm to amplify luminescence generated in the reaction of the reagent with the sample;
a filter transmitting ultraviolet light only in a wavelength range of 320 to 370 nm;
a luminescent photographic apparatus photographing a photoluminescence generated by the amplification of the luminescence; and
an image analyzing apparatus analyzing the photographed photoluminescent image.

8. An apparatus for analyzing an ATP capable of measuring one selected from the group consisting of a presence of, an amount of, and a position of an ATP in a sample, and combinations thereof, according to the method of claim 3, and comprising:
a sample-treating region in which a sample is treated such that an ATP of a sample can chemically react;
a reagent-feeding region through which a reagent that reacts with an ATP in a sample to generate luminescence is provided to the sample on the sample-treating region;
a lighting region generating ultraviolet light in a wavelength range of 320 to 370 nm to amplify luminescence generated in the reaction of the reagent with the sample;
a filter transmitting ultraviolet light only in a wavelength range of 320 to 370 nm;
a luminescent photographic apparatus photographing a photoluminescence generated by the amplification of the luminescence; and
an image analyzing apparatus analyzing the photographed photoluminescent image.

9. An apparatus for analyzing an ATP capable of measuring one selected from the group consisting of a presence of, an amount of, and a position of an ATP in a sample, and combinations thereof, according to the method of claim 4, and comprising:
a sample-treating region in which a sample is treated such that an ATP of a sample can chemically react;
a reagent-feeding region through which a reagent that reacts with an ATP in a sample to generate luminescence is provided to the sample on the sample-treating region;
a lighting region generating ultraviolet light in a wavelength range of 320 to 370 nm to amplify luminescence generated in the reaction of the reagent with the sample;
a filter transmitting ultraviolet light only in a wavelength range of 320 to 370 nm;
a luminescent photographic apparatus photographing a photoluminescence generated by the amplification of the luminescence; and
an image analyzing apparatus analyzing the photographed photoluminescent image.
